(19) United States
(12) Patent Application Publication
(10) Pub. No.: US 2002/0136771 A1
(43) Pub. Date: Sep. 26, 2002

(54) Stabilized ascorbic acid composition

(75) Inventors: James Parr, San Juan Capistrano, CA (US); Ziming Sun, Fountain Valley, CA (US); Yi Hsin Chang, Santa Ana, CA (US); Uyen Uyen Ho, Garden Grove, CA (US); Darcy George, Coto De Caza, CA (US)

Correspondence Address: JEFFER, MANGELS, BUTLER & MARMARO LLP, TENTH FLOOR, 2121 AVENUE OF THE STARS, LOS ANGELES, CA, 90067, US

(73) Assignee: AMITEE COSMETICS, INC.

(21) Appl. No.: 10/017,768

(22) Filed: Dec. 12, 2001

Related U.S. Application Data
(63) Continuation-in-part of application No. 09/224,005, filed on Dec. 31, 1998.

(51) Int. Cl. A61K 31/375; A61K 9/14

(52) U.S. Cl. 424/488; 514/474

(57) ABSTRACT

A composition including (a) about 2 to about 25 wt % of L-ascorbic acid, (b) about 0.1 to about 10 wt % of at least one selected form the group consisting of cationic polymers and cationic surfactants, and (c) about 0.1 to about 70 wt % of at least one selected from the group consisting of humectants, polymers with humectant properties, and inorganic driers. The ratio of ingredient (a) to ingredient (b) is about 1:1 to about 50:1. The composition is stable when stored at room temperature for a period of at least ten weeks.

This application is a continuation-in-part of U.S. application Ser. No. 09/224005, filed Dec. 31, 1998.

### FIELD OF THE INVENTION

The present invention relates to compositions for topical application that include ascorbic acid as an active ingredient.

### BACKGROUND OF THE INVENTION

Ascorbic acid, or Vitamin C (normally in the L-form) has been found to have three major biological functions with respect to the skin: to stimulate collagen synthesis in human skin fibroblasts; to act as an anti-oxidant to combat oxygen free redicals, which are stimulated by exposure to UV light, tobacco smoke and other environmental insults; and to act as an anti-inflammatory agent to control the inflammatory reaction associated with sunburn. Ascorbic acid acts to increase protein and collagen synthesis, with resulting anti-wrinkle effects, chelates with ferric ion to terminate oxygen-containing free radical processes, and prevents skin damage arising from excessive exposure to sunlight.

L-ascorbic acid is an alpha-ketolactone having the structure shown in FIG. 1. The number 2 and 3 carbons are double-bonded. L-ascorbic acid is stable in the solid state at ambient conditions. However, it is unstable in solution against oxidation and light degradation. Only a few days are required for a 5% ascorbic acid solution to completely decay into its oxidized form. In aqueous solution, L-ascorbic acid contains an ionizable hydrogen (pK₁=4.17 at room temperature). About 2.4% of ascorbic acid is deprotonated in a 2% aqueous solution at 25° C.

Ascorbic acid is also a moderate reducing agent, since its redox potential is only +0.127 V at pH 5.0. The oxidation reaction of ascorbic acid is shown given in FIG. 1. Ascorbic acid lose two electrons to form the dehydrated form.

Two moles of protons are involved in the half-reaction; therefore the potential (electromotive force) of the half-reaction is pH dependent.

According to the Nernst equation,

$$E = {E}^{0} + ( \frac{RT}{nF} ) ln ( \frac{{[ Vc ( ox ) ] [ {H}^{+} ]}^{2}}{[ Vc ( red ) ]} )$$

(in which R is the ideal gas constant, F is the Faraday constant, E⁰ is the standard electromotive force, [Vc(ox)] is the concentration of oxidized ascorbic acid and [Vc(red)] is the concentration of unoxidized ascorbic acid in the solution), potential is inversely proportional to the pH of the aqueous ascorbic acid solution. In the other words, when the pH is increased, the potential decreases and the stability of ascorbic acid decreases. At pH 2 and 7, the calculated potentials E are 0.305 and 0.01 V, respectively, where [Vc(ox)] and [Vc(red)] are assumed to be 1 mol L⁻¹ at 298.32 K. A plot of the pH dependence of the electromotive force for an ascorbic acid solution is given in FIG. 2. It is apparent that the deprotonated ascorbate anion is more susceptible to oxidation.

In view of the beneficial chemical and physical properties of ascorbic acid, many attempts have been made to stabilize ascorbic acid for topical application. The esterification of ascorbic acid was developed in early 1971. Stable derivatives of ascorbic acid were obtained by esterification. However, decreased activity was also observed. Ester derivatives of ascorbic acid have been used as active ingredients in skin care creams as well as hair care products. A major disadvantage of such derivatives is their loss of antioxidative effectiveness due to the inability of the ester oxygen to chelate ferric ion.

Another approach involved stabilizing ascorbic acid in aqueous solution for topical application. Two different strategies have been developed for stabilizing either anionic ascorbate at pH >4 or the unprotonated form of ascorbic acid. U.S. Pat. Nos. 2,400,171 and 2,585,580 disclose that with monothioglycerol, calcium cation or zinc cation as well as calcium-aliphatic thiocarboxylic acid at pH >4, stable compositions can be obtained. However, the anionic ascorbate is unfavorable for penetrating the skin, and is also more unstable than the un-ionized form for topical application because of the higher potential of the ascorbate anion (see FIG. 2).

Current research efforts are directed to stabilization of the unionized form of ascorbic acid for topical application. U.S. Pat. Nos. 5,736,567, 5,703,122 and 5,691,378 disclose that ascorbic acid can be stabilized over time by limiting water activity to below 0.85 using polyols. Didier and Nathalie (U.S. Pat. Nos. 5,629,004 and 5,552,446) also disclose incorporating ascorbic acid solution into a water-in-oil (w/o) emulsion to extend the life span of ascorbic acid.

The instability of ascorbic acid in aqueous solution is due to its structural alpha-keto frame, its interaction with water, and also due to oxygen permeating into the solutions. Stabilization of the anionic form or reduction of the water activity are insufficient to afford the desired stability. In order to adequately increase the stability of ascorbic acid in aqueous solution for topical application, all of the foregoing effects must be taken into consideration.

A continuing need exists for a stabilized ascorbic acid composition suitable for topical application.

### SUMMARY OF THE PREFERRED EMBODIMENTS

In accordance with one aspect of the present invention, there is provided a composition that includes (a) about 2 to about 25 wt % of L-ascorbic acid, (b) about 0.1 wt % to about 10 wt % of at least one selected form the group consisting of cationic polymers and cationic surfactants, and (c) about 0.1 wt % to about 70 wt % of at least one selected from the group consisting of humectants, polymers with humectant properties and inorganic driers. In the composition, the ratio of ingredient (a) to ingredient (b) is about 1:1 to about 50:1. The composition is stable when stored at room temperature for a period of at least ten weeks.

Emulsions containing the inventive compositions in the water phase are also provided, as are methods for preparing the inventive compositions.

Other objects, features and advantages of the present invention will become apparent to those skilled in the art from the following detailed description. It is to be understood, however, that the detailed description and specific examples, while indicating preferred embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

The invention may be more readily understood by referring to the accompanying drawings in which

FIG. 1 is an illustration of two chemical reactions for ascorbic acid in aqueous solution.

FIG. 2 is a plot of the pH dependence of the electromotive force of an ascorbic acid solution, calculated using the Nernst equation, wherein [Vc(ox)] and [Vc(red)] were assumed to be 1.0 mol L⁻¹.

FIG. 3 is a plot of concentration (wt %) vs. time (weeks) for ascorbic acid solutions according to Examples 1, 6, 11 and 16 herein at 25° C. (open symbols) and 40° C. (closed symbols), respectively, wherein the concentrations were obtained by calorimetric analysis.

### DETAILED DESCRIPTION OF THE PREFERRED EMBODIMENTS

The present invention provides stable ascorbic acid compositions for topical application. Particular embodiments of the invention are specially designed for cosmetic and dermatological products. In particular, the invention is directed to skin-care and skin-treatment products, as well as processes to incorporate the compositions into water-in-oil (w/o), oil-in-water (o/w), and water-in-oil-in-water (w/o/w) emulsions. By employing cationic polymers or cationic surfactants to form complexes with both unionized and ionized forms of ascorbic acid, and decreasing water activity by using humectants and/or inorganic driers so as to limit oxygen permeability into water, highly stable topical compositions are provided.

As used herein, an ascorbic acid composition is “stable” if the concentration of ascorbic acid in the composition does not decrease by more than 5% after storage at 25° C. for a period of ten (10) weeks.

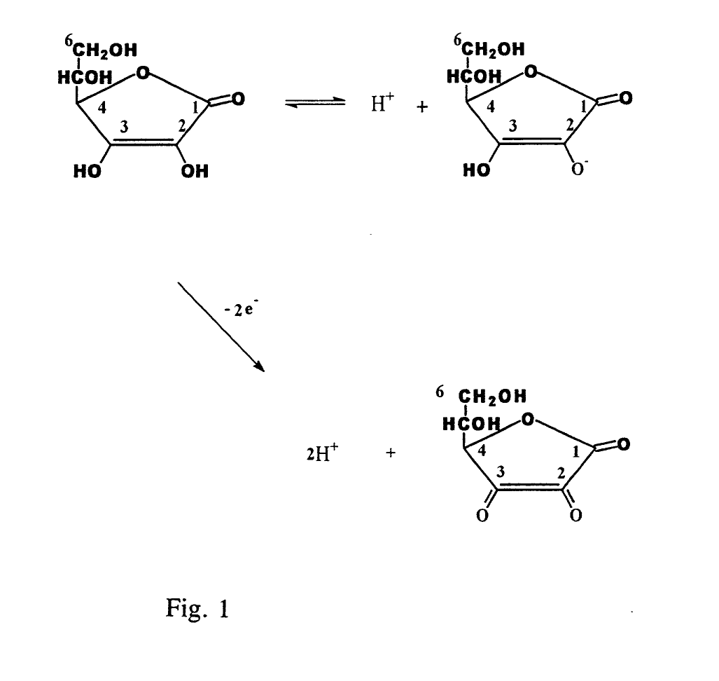

When ascorbic acid is dissolved in water in a 2% solution, about 2.4% of the ascorbic acid is ionized to form anionic ascorbate, and the ionized and unionized forms are in equilibrium (see FIG. 1). Through controlling the water activity to reduce water mobility and oxygen permeability, a more stable ascorbic acid solution is obtained. However, the ionized form is still readily oxidized. In order to stabilize the ascorbic acid solution for an extended period of time, both the ionized and unionized forms must be considered at the same time. If the ionized form is slowly oxidized over time, more of the unionized form will be ionized to maintain the equilibrium.

By introducing cationic polymers or cationic surfactants that can form complexes with both the ionized and un-ionized forms of ascorbic acid, together with humectants that reduce the water activity and the permeation of oxygen into the aqueous solution, highly stable topical compositions are produced. All of the influences on stability discussed above are taken into consideration in the inventive compositions. As a result, the inventive aqueous solutions are very stable over the course of time.

The inventive compositions are capable of stabilizing ascorbic acid over a wide range of pH (about 2 to about 7) by controlling the ratio of ascorbic acid to cationic polymers and/or cationic surfactants. As an extra beneficial feature, the inventive compositions have an elegant feeling when applied to the skin and are less irritating, making the compositions very suitable for use in skin-care and skin-treatment products.

When materials (solutes) are dissolved in water to form a diluted ideal solution, the molecules of the various species components are so similar to one another that molecules of one component can replace molecules of another component in the solution without changing the solution's energy or spatial structure. In other words, the molecules of solute and solvent resemble one another closely in size, shape and intermolecular interactions. Therefore, the activity of water molecules can be described by Raoult's Law, P_{A}=X_{A}P_{A}, where P_{A} and P_{A}* are vapor pressures for aqueous solution and pure water at constant temperature, respectively; X_{A} is water activity, X_{A}=n_{A}/(n_{A}+n_{B}), where n_{A} is the number of moles of water and n_{B} is the number of moles of solute. The water activity is determined by measuring the vapor pressure of the solution.

If, however, the size, shape and/or intermolecular interactions of water and the solute are different, then deviations from Raoult's Law will be observed. If the interaction between molecules of the solute and molecules of water is stronger than the interaction between the molecules of water, as well as stronger than that between the molecules of solute, a negative deviation from Raoult's Law will be observed. In other words, lesser amounts of solute will cause great decreases in water activity.

The humectants preferably used in the inventive compositions are able to form multiple hydrogen bonds with molecules of water. Thus, large negative deviations from Raoult's Law are usually observed in aqueous solutions including the humectants. The decreased water activity results in less mobility of free water molecules and decreased oxygen permeability into the water.

Exemplary cationic polymers and cationic surfactants that are usefully employed in compositions according to the invention include, without limitation, polyquats such as polyquaternium 2, polyquaternium 4, polyquaternium 6, polyquaternium 7, polyquaternium 10, polyquaternium 11, polyquaternium 16, polyquaternium 17, polyquaternium 18, polyquaternium 22, polyquaternium 24, polyquaternium 27, polyquaternium 28, polyquaternium 39, polyquaternium 42 and polyquaternium 46 and mixtures thereof.

Quaternized cellulose, collagens and proteins are also cationic polymers that are beneficially employed in embodiments of the inventive compositions. These include, without limitation, guar hydroxypropyltrimonium chloride, lauryidimonium hydroxypropyl oxyethyl cellulose, lauryidimonium hydroxyethyl cellulose, lauryldimonium hydroxypropyl hydrolyzed silk, protonated polyethylenimine, hydrolyzed casein, cocodimonium hydroxypropyl oxyethyl cellulose, cocodimonium hydroxypropyl hydrolyzed collagen, cocodimonium hydroxypropyl hydrolyzed hair keratin, cocodimonium hydroxypropyl hydrolyzed keratin, cocodimonium hydroxypropyl hydrolyzed wheat protein, stearyidimonium hydroxyethyl cellulose, stearyidimonium hydroxypropyl hydrolyzed collagen, stearyidimonium hydroxypropyl hydrolyzed wheat protein, stearyldimonium hydroxypropyl oxyethyl cellulose, stearyltrimonium hydroxyethyl hydrolyzed collagen, lauryl methyl gluceth-10 hydroxypropyidimonium chloride, oleyl betaine and cocamidopropyl betaine.

Cationic surfactants that can be employed according to the invention include, without limitation, cetrimonium chloride, cetrimonium bromide, dicetyldimonium chloride, acetamidopropyl trimonium chloride, behentrimonium chloride, hydroxyethyl cetyldimonium chloride, cetalkonium chloride and mixtures thereof.

Humectants and/or inorganic driers beneficially employed in embodiments of the inventive composition include, without limitation, glycerol, sorbitol, panthenol, 1,2-propylene glycol, 1,3-butylene glycol, sodium lactate, potassium lactate, magnesium lactate, calcium chloride, magnesium sulfate and mixtures of thereof.

Polymers with humectant properties are useful in combination with cationic polymers and/or cationic surfactants. Examples of useful polymers with humectant properties include, without limitation, PVP/MA copolymer, PVP/MA decadiene crosspolymer, PVP, PVP/dimethylaminoethyl methacrylate copolymer, PVP/dimethylaminoethyl methacrylate/polycarbamyl polyglycol ester, PVP/dimethiconylacrylate/polycarbamyl polyglycol ester, PVP/polycarbamyl polyglycol ester, PVP/VA copolymer, hydroxyethyl cellulose, hydroxypropyl guar, hydroxypropylmethyl cellulose, hydroxypropyl cellulose, polyethylene glycol 200, polyethylene glycol 300, polyethylene glycol 400, polyethylene glycol 600, polyethylene glycol 900, polyethylene glycol 1000, polyethylene glycol 1450, methyl glucose sesquiisostearate, methyl gluceth-10, methyl gluceth-20, PPG-10 methyl glucose ether, PPG-20 methyl glucose ether, PPG-20 methyl glucose ether distearate, and mixtures thereof.

In preferred embodiments of the inventive composition, metal sequestering agents, such as salts of EDTA, are also used for chelating ferric and manganese ions that are often present in water, since both of these ions are capable of accelerating the oxidation of ascorbic acid.

Preferred embodiments of the inventive composition are multiphase emulsions, such as w/o, o/w and w/o/w emulsions. Exemplary oil components for multiphase emulsion embodiments include, without limitation, silicone oils, mineral oil, peanut oil, soy oil, olive oil, wheat germ oil, avocado oil, castor oil, Jojoba oil and synthetic esters by direct reaction of fatty acids with alcohols. More specific examples of synthetic esters include isopropyl esters, ethxylhexyl esters, oleic acid esters, caprylic/capric acid esters, N-butyl stearate, isocetyl stearate, octyidodecanol, diisopropyl adipate and pentaerythritol tetraisostearate.

Emulsifiers and stabilizers useful in embodiments of the inventive composition include, without limitation, sodium cocyl isothionate, potassium cetyl phosphate, ethoxylate fatty alcohol, PEG esters of fatty acids, ethoxylated sorbitan esters, ethoxylated monoglycerides, ethoxylated castor oil derivatives, sorbitan monooleate, sorbitan sesquioleate, glycerol monooleate, polyglycerol esters, oleate, ricinoleates, isostearate, methoxy PEG-22 dodecyl glycol copolymer, lanolin derivatives, polyacrylate resins, acrylamide/sodium acrylate copolymer and PEG-45 dodecyl glycol copolymer.

The quantity of cationic polymers and/or cationic surfactants used in the inventive composition is about 0.1 wt % to about 10 wt %, preferably 0.2 wt % to 5 wt % depending on the specific individual cationic polymer and/or cationic surfactant, more preferably 0.3 wt % to 2 wt % for polyquaternium 6 as the cationic polymer. The quantity of humectant(s) is about 1 wt % to about 70 wt %, preferably 5 wt % to 40 wt %, more preferably 20 wt % to 30 wt % when polyquaternium 6 is used in the inventive composition. The quantity of polymer(s) with humectant properties used to assist the cationic polymers and/or cationic surfactants is about 0.1 wt % to about 15 wt %, preferably 0.5 wt % to 5 wt %, more preferably 0.5 wt % to 1 wt % when polyquaternium 10 (JR 400) is used. When inorganic driers are used, the quantity thereof is about 0.1 wt % to about 1 wt %, preferably 0.2 wt % to 1 wt %, more preferably 0.2 wt % to 0.3 wt %. The amount of sequstering agent, when used, is preferably less than 0.1 wt %. The oil phase in w/o, o/w and w/o/w emulsion embodiments of the inventive composition preferably constitutes about 15% to about 50%, depending upon the type of emulsion.

A classical colorimetric assay (Day et al, Clin. Biochem. 1979, 1 2, pp22-26) has been used for determining the content of the ascorbic acid in aqueous compositions over the time. The assay is based upon the quantitative reduction of the ferric complex of 2, 4, 6-tripyridyl-s-triazine by ascorbic acid to a purple colored ferrous complex of 2, 4, 6-tripyridyl-s-triazine in a buffer solution of acetic acid-acetate. As the reaction takes place, the solution changes color from almost colorless to purple, which is detected at 595 nm on a UV spectrophotometer. The oxidized dehydro ascorbic acid does not react with ferric complexes of 2, 4, 6-tripyridyl-s-triazine and does not interfere with the determination.

The invention is further illustrated by the following non-limiting examples. The composition solutions are stored in light-impermeable bottles or jars for stability tests. The ascorbic acid content is determined by colorimetry over time for ten weeks at both room temperature and 40° C. Stability data are set forth in Table 1.

### EXAMPLE 1

d.i. water 25.94%

polyquternium-6 (40%) 2.17%

tetrasodium EDTA 0.05%

L-ascorbic acid 2.0%

10% sodium hydroxide 4.54%

panthenol 0.3%

propylene glycol 65%

The pH is 6.65 at room temperature

### EXAMPLE 2

d.i. water 23.47%

polyquternium-24 0.5%

tetrasodium EDTA 0.05%

L-ascorbic acid 5.0%

10% sodium hydroxide 5.68%

panthenol 0.3%

butylene glycol 65%

The pH is 4.95 at room temperature

### EXAMPLE 3

d.i. water 30.65%

cocodimonium hydroxypropyl

oxyethyl cellulose 4.0%

tetrasodium EDTA 0.05%

panthenol 0.3%

L-ascorbic acid 5.0%

butylene glycol 60%

The pH is 3.12 at room temperature.

| | |
|---|---|
| EXAMPLE 4 | |
| d.i. water | 25.59% |
| acetamidopropyl | 2.21% |
| trimonium chloride | |
| disodium EDTA | 0.05% |
| L-ascorbic acid | 2.0% |
| 10% sodium hydroxide | 4.45% |
| panthenol | 0.3% |
| butylene glycol | 65% |
| The pH is 6.39. | |
| EXAMPLE 5 | |
| d.i. water | 27.36% |
| polyquaternium-2 | 1.5% |
| tetrasodium EDTA | 0.05% |
| PVP/VA | 2.0% |
| panthenol | 0.3% |
| ascorbic acid | 5.0% |
| 10% sodium hydroxide | 3.785% |
| propylene glycol | 60% |
| The pH is 4.55. | |
| EXAMPLE 6 | |
| d.i. water | 29.15% |
| polyquqternium-10 (JR-30M) | 0.5% |
| tetrasodium EDTA | 0.05% |
| panthenol | 0.3% |
| L-ascorbic acid | 5.0% |
| propylene glycol | 65% |
| The pH is 3.21. | |

Polyquaternium-10 (0.5 g) is dissolved in 29.15 g d.i. water and the solution is warmed to 60° C. for hydration. The solution is cooled to room temperature and the other ingredients are added.

| | |
|---|---|
| EXAMPLE 7 | |
| d.i. water | 66.95% |
| disodium EDTA | 0.05% |
| polyquaternium-11 | 2.0% |
| sorbitol | 5.0% |
| panthenol | 0.5% |
| L-ascorbic acid | 10.0% |
| PEG-4 | 15% |
| magnesium sulfate | 0.5% |
| The pH is 2.06. | |
| EXAMPLE 8 | |
| d.i. water | 32.73% |
| polyquaternium-10 (JR-400) | 0.4% |
| PVP | 1.5% |
| disodium EDTA | 0.07% |
| panthenol | 0.3% |
| L-ascorbic acid | 15% |
| propylene glycol | 50% |
| pH at 2.46. | |
| EXAMPLE 9 | |
| d.i. water | 32.93 |
| polyquaternium-10 (JR-125) | 0.2% |
| PVP | 1.5% |
| disodium EDTA | 0.07% |
| L-ascorbic acid. | 15.0% |
| panthenol | 0.3% |
| propylene glycol | 35% |
| PEG-4 | 15% |
| The pH is at 2.51. | |
| EXAMPLE 10 | |
| d.i. water | 59.63% |
| hydroxypropyl guar | 0.3% |
| hydroxypropyltrimonium chloride | |
| disodium EDTA | 0.07% |
| PVP [poly(vinylpyrrolidone)] | 0.7% |
| L-ascorbic acid | 15% |
| panthenol | 0.3% |
| glycerin | 15% |
| PEG-4 | 10% |
| The pH is 2.20. | |

0.3 g of hydroxypropyl guar hydroxypropyltrimonium chloride is added to 50 g of d.i. water and hydrated by heating the solution to 60° C. After the solution is cooled to room temperature, the other ingredients are added.

| EXAMPLE 11 | |
|---|---|
| d.i. water | 64.63% |
| disodium EDTA | 0.07% |
| polyquaternium-27 | 3.0% |
| PVP | 2.0% |
| panthenol | 0.3% |
| L-ascorbic acid | 20.0% |
| PEG-4 | 10.0% |
| The pH is 2.07. | |
| EXAMPLE 12 | |
| A. L-ascorbic acid | 1% |
| propylene glycol | 99% |
| B. L-ascorbic acid | 1% |
| Ethoxydiglycol | 99% |
| EXAMPLE 13 | |
| d.i. water | 74.95% |
| disodium EDTA | 0.05% |
| stearyldimonium hydroxypropyl | 15% |
| oxyethyl cellulose (20-24%) | |
| L-ascorbic acid | 10% |
| pH is 2.28 at room temperature. | |
| EXAMPLE 14 | |
| d.i. water | 94.95% |
| disodium EDTA | 0.05% |
| polyquaternium-10 (JR-400) | 0.1% |
| L-ascorbic acid | 0.5% |
| pH is 2.85 at room temperature. | |
| EXAMPLE 15 | |
| Water phase | |
| d.i. water | 29.23% |
| disodium EDTA | 0.058% |
| PEG-4 | 12.42% |
| polyquaternium-28 | 2.65% |
| L-ascorbic acid | 12.42% |
| butylene glycol | 12.42% |
| panthenol | 0.3% |
| Silicone oil phase | |
| Dow Corning 3225 fluid ™ | 10.0% |
| (cyclomethicone, dimethicone | |
| copolyol) | |
| gel aid 8717 ™ | 15% |
| (cyclomethicone, cyclomethicone, | |
| dimethicone) | |
| Rhodorsil 45v5 ™ | 5% |
| (cyclomethicone) | |
| PPG-3 myristyl ether | 0.5% |

The water phase is added to the oil phase slowly with turbulent mixing. A gel-like composition is formed.

| EXAMPLE 16 (oil in water) | |
|---|---|
| water phase | |
| d.i. water | 73.25% |
| disodium EDTA | 0.05% |
| salicylic acid | 0.30% |
| polyquaternium-4 | 1.00% |
| L-ascorbic acid | 5.00% |
| glycerin | 3.00% |
| panthenol | 0.30% |
| sepigel | 1.50% |
| sodium cocoyl isethionate | 1.0% |
| oil phase | |
| stearyl alcohol | 1.5% |
| cetyl alcohol | 1.0% |
| steareth-21 | 1.25% |
| steareth-20. | 1.25% |
| PEG-100 stearate | 0.5% |
| PEG-20 | 1.0% |
| PPG-15 stearyl ether | 8.0% |
| Fragrance | 0.1% |
| EXAMPLE 17 | |
| phase A | |
| d.i. water | 13.12% |
| disodium EDTA | 0.01% |
| polyquaternium-6 | 0.20% |
| magnesium sulfate | 0.06% |
| L-ascorbic acid | 3.9% |
| butylene glycol | 3.0% |
| panthenol | 0.12% |
| phase B | |
| Dow Corning 200 fluid 50 cts | 0.9% |
| (dimethicone) | |
| Dow Corning 246 (cyclomethcone) | 0.9% |
| gel aid 8717 ™ (cyclomethicone, | 1.05% |
| cyclomethicone, dimethicone) | |
| Rhodorsil 45v5 ™ | 5.85% |
| (cyclomethicone) | |
| Abil EM-97 | 0.9% |
| (dimethicone copolyol, | |
| cyclopentasiloxane) | |
| phase C | |
| Add phase A into phase B | |
| slowly to form w/o phase | |
| Sepigel | 3.0% |
| phase D | |
| d.i. water | 46.5% |
| butylene glycol | 20.0% |
| polysorbate-20 | 0.5% |

Add Sepigel into w/o phase slowly and add phase D into phase C rapidly. The stable triple phase can be seen under a microscope and detected by conductivity measurement.

Examples 1-6 explore the pH-dependent stability of the composition by keeping other ingredients at almost constant levels. To simplify the examination, only one cationic surfactant (or one cationic polymer) and one humectant are used in each formula.

Two results are observed from the stability data obtained at different pH:

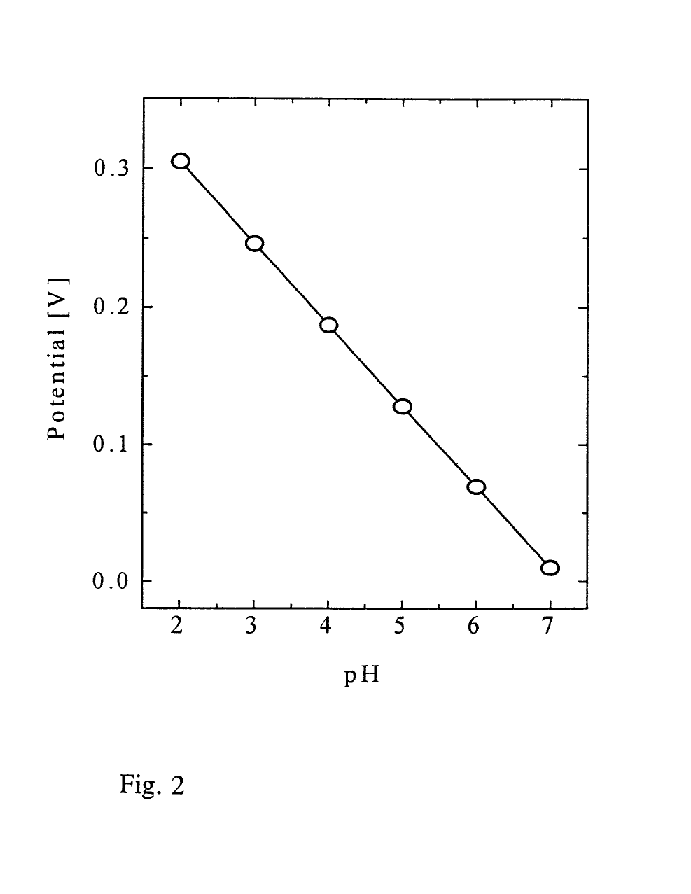

(1) More degradation is observed in high pH solutions than in low pH solutions. But overall, after two month, only a slight degradation of ascorbic acid was observed for these compositions at room temperature. The experimental data are in agreement with the plot of pH-dependent electromotive force shown in FIG. 2.

(2) Three different cationic polymers or cationic surfactants were used to stabilize the ascorbic acid solutions. The stability data show that they profoundly increase the stability of the ascorbic acid in aqueous solution. The experimental data are also in agreement with the plot of pH-dependent electromotive force shown in FIG. 2.

Examples 7-11 explore the possibility of using high concentrations of ascorbic acid solution and also varying the water content in the compositions. All compositions are very stable at room temperature; the extent of degradation observed from the stability data over eight weeks is negligible.

Examples 12(A) and 12(B) test polyols to stabilize the ascorbic acid alone. As the data in Table 1 show, noticeable degradation (more than 10%) is evident over the course of one month at room temperature. The results indicate that polyols by themselves can stabilize ascorbic acid, but cannot provide very stable compositions.

For comparison, example 13 tests the stability of ascorbic acid aqueous solution with a cationic polymer alone. The stearyidimonium hydroxypropyl oxyethyl cellulose is the only agent for stabilizing the ascorbic acid. The stability data at room temperature and at 40° C. for two weeks are very close, and only slight degradation is observed for the sample. The results strongly suggest that the cationic polymer forms complexes with ascorbic acid to stabilize the ascorbic acid in aqueous solution. Since both the cationic polymers and humectants are able to stabilize the ascorbic acid aqueous solution to a great degree, compositions containing both of these components are optimal for stabilizing ascorbic acid in aqueous solution.

Example 14 illustrates a solution having lower concentrations of ascorbic acid and cationic polymers. The ascorbic acid degraded more rapidly than in other examples (see Table 1). About 20% of the ascorbic acid oxidized at room temperature in two weeks.

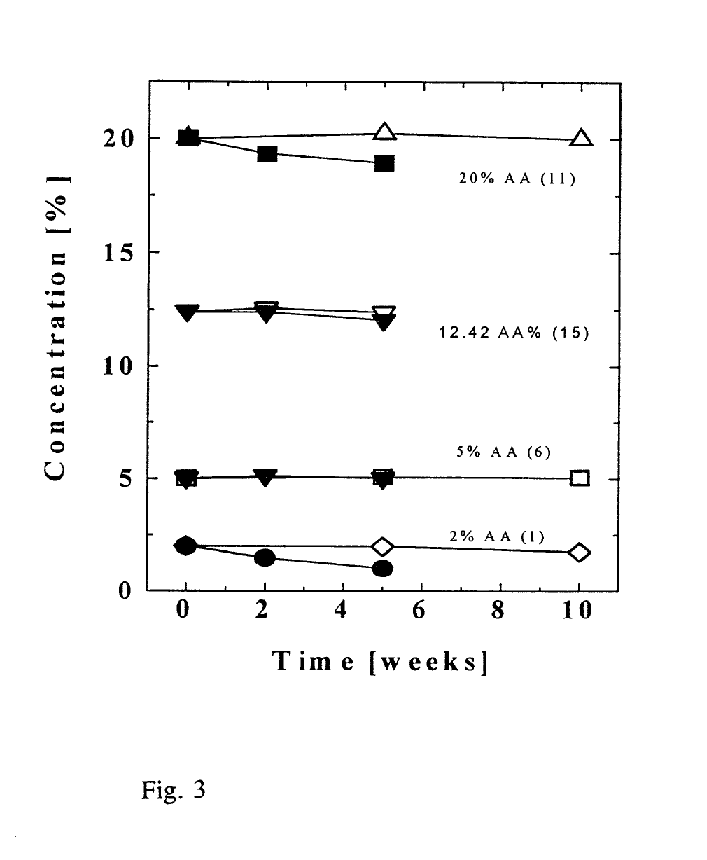

The stability at 40° C. is also determined for seven other examples (listed in Table 1). The data for examples 1, 6, 11 and 16 are plotted in FIG. 3. In FIG. 3, room temperature data (open symbols) and data at 40° C. (filled symbols) are plotted together for comparison. At 40° C., the apparent degradation is noticeable for example 1. This is attributable to the relatively high pH of the cmposition (pH 6.65). Slight degradation is found for example 11, which is attributable to the lower humectant content. Insignificant degradation is observed for example 6 at both room temperature and 40° C., which may be due to the combination of a large amount of humectants and cationic polymers.

The water-in-oil (w/o), oil-in-water (w/o) and water-in-oil-in-water (w/o/w) emulsions are also designed for further increasing the stability of ascorbic acid solution for topical application. For all of these examples, unnoticeable degradation is observed even at 40° C. for two weeks. When a stable ascorbic acid solution is encapsulated in the oil phase, the most stable compositions are obtained. Essentially unnoticeable degradation of ascorbic acid is observed in example 17 at 40° C. over five weeks.

TABLE 1

| Example | [A]₁ | pH | [A]_{5,RT} | [A]_{10,RT} | [A]_{2,40} | [A]_{5,40} |
|---|---|---|---|---|---|---|
| 1 | 2.0 | 6.65 | 1.98 | 1.75 | 1.48 | 1.01 |
| 2 | 5.0 | 4.95 | 4.97 | 4.52 | | |
| 3 | 5.0 | 3.12 | 5.20 | 5.15 | | |
| 4 | 2.0 | 6.39 | 1.94 | 1.89 | | |
| 5 | 5.0 | 4.55 | 4.97 | 5.01 | | |
| 6 | 5.0 | 3.21 | 5.08 | 5.05 | 5.12 | 5.00 |
| 7 | 10.0 | 2.34 | 10.16 | | 10.03 | |
| 8 | 15.0 | 2.46 | 15.20 | 15.05 | | |
| 9 | 15.0 | 2.51 | 15.10 | 15.04 | | |
| 10 | 15.0 | 2.20 | 15.21 | 15.19 | | |
| 11 | 20.0 | 2.06 | 20.24 | 20.01 | 19.32 | 18.90 |
| 12(A) | 1.0 | | 0.89 | 0.76 | | |
| 12(B) | 1.0 | | 0.85 | 0.73 | | |
| 13 | 10.0 | 2.29 | 10.12 | | 9.30 | |
| 14 | 0.5 | 2.85 | | | 0.30 | |
| 15 | 12.42 | | 12.57 | | 12.40 | 12.04 |
| 16 | 5.0 | 2.35 | 5.06 | | | |
| 17 | 3.9 | | 3.91 | | 3.80 | 3.83 |

[A]₁ = initial ascorbic acid concentration, %
[A]_{5,RT} = ascorbic acid concentration after 5 weeks, room temperature
[A]_{10,RT} = ascorbic acid concentration after 10 weeks, room temperature
[A]_{2,40} = ascorbic acid concentration after 2 weeks, 40° C.
[A]_{5,40} = ascorbic acid concentration after 5 weeks, 40° C.

What is claimed is:

1. A stabilized ascorbic acid composition comprising:
(a) about 2 to about 25 wt % of L-ascorbic acid,
(b) about 0.1 to about 10 wt % of at least one selected from the group consisting of non-film forming cationic polymers and cationic surfactants, and
(c) about 0.1 to about 70 wt % of at least one selected from the group consisting of humectants, polymers with humectant properties, and inorganic driers,
wherein the ratio of ingredient (a) to ingredient (b) is about 0.02:1 to about 1:1, and wherein said composition is stable when stored at room temperature for a period of at least ten weeks.

2. The composition of claim 1 wherein ingredient (b) forms complexes with both the ionized and the non-ionized forms of L-ascorbic acid.

3. The composition of claim 1 which is stable at a pH from about 2 to about 7 at room temperature.

4. The composition of claim 1 wherein said cationic surfactant is selected from the group consisting of alkyltrimonium chloride and alkyltrimonium bromide.

5. The composition of claim 1 wherein said cationic polymers are water-soluble.

6. The composition of claim 1 wherein said cationic polymers are selected from the group consisting of guar cationic gums, cationic collagens, cationic keratins, cationic celluloses and cationic hydrolyzed proteins.

7. The composition of claim 1 said composition displays a negative deviation from Raoult's Law.

8. The composition of claim 1 wherein said humectant is an organic molecule having a plurality of hydroxyl groups.

9. The composition of claim 1 wherein said humectant forms multiple hydrogen bonds such that said composition displays a negative deviation from Raoult's Law.

10. The composition of claim 1 wherein said inorganic drier forms stable water-metal ion complexes.

11. The composition of claim 1 wherein said polymer with humectant properties forms multiple hydrogen bonds with water molecules thereby reducing the mobility of water molecules.

12. The composition of claim 11 wherein the said polymer with humectant properties also forms multiple hydrogen bonds with ascorbic acid molecules.

13. The composition of claim 1 wherein said polymer with humectant properties is selected from the group consisting of polyoxyethylene glycol, poly(vivylpyrrolidone), poly(vinylpyrrolidone) copolymers, cellulose and cellulose derivatives.

14. The composition of claim 1 wherein the concentration of said polymer with humectant properties is about 0.1 wt % to about 20 wt %.

15. The composition of claim 1 further comprising a metal sequestering agent.

16. An emulsion comprising:
(i) an oil phase, and
(ii) a water phase comprising the composition of claim 1.

17. The emulsion of claim 16 wherein said oil phase comprises one selected from the group consisting of an oil, an emulsifier and mixtures thereof.

18. The composition of claim 16 which is a water-in-oil (w/o) emulsion.

19. The composition of claim 16 which is an oil-in-water (o/w) emulion.

20. The composition of claim 16 which is a water-in-oil-in-water (w/o/w) emulsion.

21. The composition of claim 20 wherein the oil phase of said w/o/w emulsion comprises a silicone oil, and wherein the inner water phase of said w/o/w emulsion comprises said ingredients (a)-(c).

22. The composition of claim 21 wherein the concentration of ascorbic acid in said inner water phase is about 1 % to about 7%.

23. The composition of claim 21 wherein the outer water phase comprises sepigel gel emulsifier.

24. The composition of claim 23 wherein said oil phase comprises a silicone oil and a silicone oil copolyol.

25. A method of making an L-ascorbic acid composition that is stable when stored at room temperature for a period of at least two months, said method comprising the step of combining
(a) about 2 to about 25 wt % of L-ascorbic acid,
(b) about 0.1 to about 10 wt % of at least one selected from the group consisting of non-film forming cationic polymers and cationic surfactants, and
(c) about 0.1 to about 70 wt % of at least one selected from the group consisting of humectants, polymers with humectant properties, and inorganic driers,
wherein the ratio of ingredient (a) to ingredient (b) is about 0.02:1 to about 1:1.
